(19) United States
(12) Patent Application Publication
(10) Pub. No.: US 2019/0290871 A1
(43) Pub. Date: Sep. 26, 2019

(54) TRACHEAL TUBE

(71) Applicant: Tianjin Medis Medical Device Co., Ltd., Tianjin (CN)

(72) Inventor: Dewei ZOU, Tianjin (CN)

(21) Appl. No.: 16/441,043

(22) Filed: Jun. 14, 2019

Related U.S. Application Data
(63) Continuation-in-part of application No. PCT/CN2017/082823, filed on May 3, 2017.

(30) Foreign Application Priority Data
Dec. 14, 2016 (CN) 201621368973.1

(51) Int. Cl. A61M 16/04; A61L 29/06

(52) U.S. Cl. CPC A61M 16/0486; A61M 16/044; A61M 2025/1061; A61L 29/06; A61M 16/0463

(57) ABSTRACT

A tracheal tube including: a catheter, a suction tube, a gas-charging tube, and an inflatable balloon. The catheter includes a first portion and a second portion. The first portion includes a first hole and a second hole, and the second portion includes a third hole and a fourth hole. The gas-charging tube includes a first end and a second end. The suction tube includes a first end and a second end. The inflatable balloon is disposed on the second portion of the catheter. The inflatable balloon includes a flange, and the inflatable balloon is fixed on the second portion via the flange. The first end of the gas-charging tube is disposed outside the catheter, and the second end of the gas-charging tube enters the catheter via the first hole and extends to connect to the third hole. The first end of the suction tube is disposed outside the catheter.

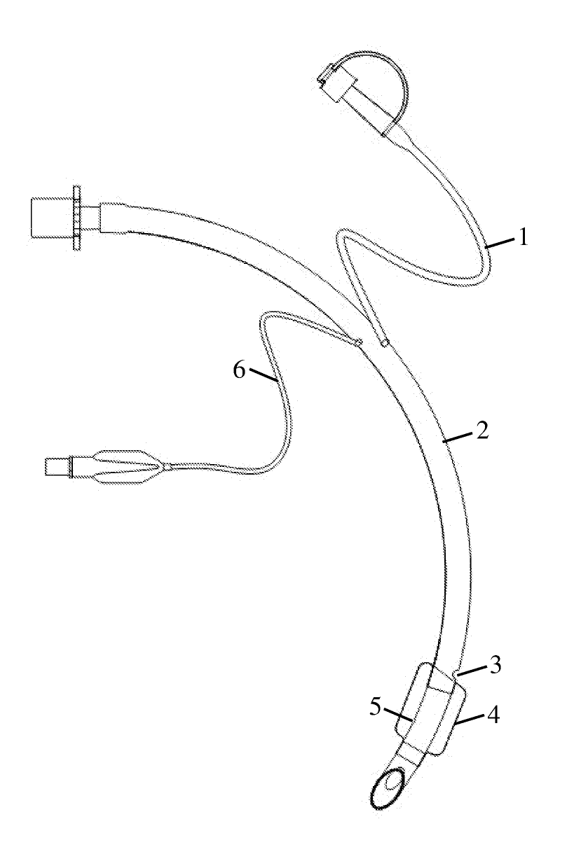

### CROSS-REFERENCE TO RELATED APPLICATIONS

This application is a continuation-in-part of International Patent Application No. PCT/CN2017/082823 with an international filing date of May 3, 2017, designating the United States, now pending, and further claims foreign priority benefits to Chinese Patent Application No. 201621368973.1 filed Dec. 14, 2016. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND

This disclosure relates to a tracheal tube.

A tracheal tube is a catheter inserted into the trachea for the primary purpose of establishing and maintaining a patient airway to ensure the adequate exchange of oxygen and carbon dioxide.

Conventional tracheal tubes include a catheter, an inflatable balloon, a gas-charging tube and a suction tube. The inflatable balloon is spherical or ellipsoidal and directly bonded to the catheter. The catheter includes a side hole communicating with the suction tube. The side hole is spaced from the inflatable balloon. Thus, the exudates between the side hole and the inflatable balloon cannot be removed.

### SUMMARY

Disclosed is a tracheal tube that is efficient in removing exudates out of the trachea.

Disclosed is a tracheal tube comprising: a catheter, a suction tube, a gas-charging tube, and an inflatable balloon. The catheter comprises a first portion and a second portion. The first portion comprises a first hole and a second hole, and the second portion comprises a third hole and a fourth hole.

The gas-charging tube comprises a first end and a second end. The suction tube comprises a first end and a second end. The inflatable balloon is disposed on the second portion of the catheter.

The inflatable balloon comprises a flange, and the inflatable balloon is fixed on the second portion via the flange. The first end of the gas-charging tube is disposed outside the catheter, and the second end of the gas-charging tube enters the catheter via the first hole and extends to connect to the third hole. The first end of the suction tube is disposed outside the catheter, and the second end of the suction tube enters the catheter via the second hole and extends to connect to the fourth hole. The third hole is located in and communicate with the inflatable balloon. The fourth hole is disposed outside the inflatable balloon. The inflatable balloon is sheathed on the catheter to form a first surface and a second surface. The first surface is between the third hole and the fourth hole. The first surface is oriented to be non-perpendicular to a central line of the catheter; and the fourth hole is adjacent to the first surface, and no gap exists between the fourth hole and the first surface.

The catheter can have an outer diameter of between 3 and 15 mm, and comprise a biomedical polymer material.

The biomedical polymer material can be polyvinyl chloride or polyurethane.

The fourth hole can have an outer diameter of between 2 and 15 mm.

Advantages of the tracheal tube as described in the disclosure are summarized as follows. The exudates are accumulated on the titled surface of the inflatable balloon, and there is no gap between the fourth hole (the suction hole) and the titled surface. Thus, the exudates can be completely removed out of the trachea.

### BRIEF DESCRIPTION OF THE DRAWINGS

The invention is described hereinbelow with reference to accompanying drawings, in which the sole FIGURE is a schematic diagram of a tracheal tube as described in the disclosure.

In the drawings, the following reference numbers are used: 1. Suction tube; 2. Catheter, 3. Fourth hole; 4. Inflatable balloon; 5. Third hole; 6. Gas-charging tube.

### DETAILED DESCRIPTION

To further illustrate, embodiments detailing a tracheal tube are described below. It should be noted that the following embodiments are intended to describe and not to limit the disclosure.

As shown in the sole FIGURE, provided is a tracheal tube, comprising a catheter 2, a suction tube 1, a gas-charging tube 6, and an inflatable balloon 4. The catheter 2 comprises a first portion and a second portion. The first portion comprises a first hole and a second hole, and the second portion comprises a third hole 5 and a fourth hole 3. The gas-charging tube comprises a first end and a second end. The suction tube 1 comprises a first end and a second end. The inflatable balloon 4 is disposed on the second portion of the catheter.

The inflatable balloon 4 comprises a flange, and the inflatable balloon is fixed on the second portion via the flange. The first end of the gas-charging tube 6 is disposed outside the catheter 2, and the second end of the gas-charging tube 6 enters the catheter 2 via the first hole and extends to connect to the third hole 5. The first end of the suction tube 1 is disposed outside the catheter 2, and the second end of the suction tube 1 enters the catheter 2 via the second hole and extends to connect to the fourth hole. The third hole 5 is located in and communicate with the inflatable balloon 4. The fourth hole 3 is disposed outside the inflatable balloon. The inflatable balloon 4 is sheathed on the catheter 2 to form a first surface and a second surface. The first surface is between the third hole 5 and the fourth hole 3. The first surface is oriented to be non-perpendicular to the central line of the catheter 2; and the fourth hole 3 is adjacent to the first surface, and no gap exists between the fourth hole and the first surface.

The catheter 2 has an outer diameter of between 3 and 15 mm and employs a biomedical polymer material. The biomedical polymer material is polyvinyl chloride or polyurethane. Preferably, the outer diameter is 12 mm, and the material is polyvinyl chloride.

The fourth hole 3 has an outer diameter of between 2 and 15 mm. In this embodiment, the outer diameter is 8 mm.

In use, the catheter 2 is inserted into the trachea of a patient to an appropriate depth. Thereafter, the gas is guided via the gas-charging tube 6 and the third hole 5 to the inflatable balloon 4. The inflatable balloon 4 expands, blocks the airway, and fixed the catheter. This benefits the mechanical ventilation.

In the inflated state, the upper surface of the inflatable balloon 4 is tilted relative to the central line of the catheter. That is, the upper surface is non-perpendicular to the central line of the catheter. With the progress of the mechanical ventilation, the exudates are produced in the trachea and accumulate, under the action of gravity, at the lowest point of the interface of the side wall of the second portion of the catheter 2 with the titled upper surface of the inflatable balloon 4. Because the fourth hole 3 is disposed adjacent to the upper surface and at the lowest point of the interface of the side wall of the second portion of the catheter 2 with the upper surface of the inflatable balloon 4, the exudates gather around the fourth hole 3. The suction tube 1 can extract the exudates via the fourth hole 3.

According to the clinical needs, the suction tube 1 can be connected to an external suction device, and the exudates around the fourth hole 3 are sucked into the suction tube 1 and completely discharged out of the human body through the suction tube 1.

It will be obvious to those skilled in the art that changes and modifications may be made, and therefore, the aim in the appended claims is to cover all such changes and modifications.

What is claimed is:

1. A device, comprising:
a catheter comprising a first portion and a second portion, the first portion comprising a first hole and a second hole, and the second portion comprising a third hole and a fourth hole;
a gas-charging tube comprising a first end and a second end;
a suction tube comprising a first end and a second end; and
an inflatable balloon disposed on the second portion of the catheter;
wherein:
the inflatable balloon comprises a flange, and the inflatable balloon is fixed on the second portion via the flange;
the first end of the gas-charging tube is disposed outside the catheter, and the second end of the gas-charging tube enters the catheter via the first hole and extends to connect to the third hole;
the first end of the suction tube is disposed outside the catheter, and the second end of the suction tube enters the catheter via the second hole and extends to connect to the fourth hole;
the third hole is located in and communicate with the inflatable balloon;
the fourth hole is disposed outside the inflatable balloon;
the inflatable balloon is sheathed on the catheter to form a first surface and a second surface; the first surface is between the third hole and the fourth hole;
the first surface is oriented to be non-perpendicular to a central line of the catheter; and
the fourth hole is adjacent to the first surface, and no gap exists between the fourth hole and the first surface.

2. The device of claim 1, wherein the catheter has an outer diameter of between 3 and 15 mm, and comprises a biomedical polymer material.

3. The device of claim 2, wherein the biomedical polymer material is polyvinyl chloride or polyurethane.

4. The device of claim 1, wherein the fourth hole has an outer diameter of between 2 and 15 mm.
